(19) United States
(12) Patent Application Publication
(10) Pub. No.: US 2011/0098675 A1
(43) Pub. Date: Apr. 28, 2011

(54) DEVICE FOR INJECTING A SOLID

(75) Inventor: Christian SCHMALZ, Lyss (CH)

(73) Assignee: FORTEQ NIDAU AG, Nidau (CH)

(21) Appl. No.: 12/910,092

(22) Filed: Oct. 22, 2010

(30) Foreign Application Priority Data
Oct. 23, 2009 (EP) 09 405 186.9

(51) Int. Cl. A61M 5/31

(52) U.S. Cl. 604/506; 604/60

(57) ABSTRACT

A device (100) for injecting a solid (500) into a human or animal body comprises a syringe body (300) with a first tubular interior for receiving the solid (500), a retaining device for retaining the solid (500) in the syringe body (300), and a needle (200) with a second tubular interior. The needle (200) is connected to the syringe body (300) such that the second tubular interior of the needle (200) communicates with the first tubular interior of the syringe body (300). The device further comprises a ram (400), which can be driven through the first interior into the second interior. The ram (400) can cooperate directly with the retaining device, such that the retaining device can be deactivated by means of the ram (400).

### TECHNICAL FIELD

The invention relates to a device for injecting a solid into a human or animal body, said device comprising a syringe body with a first tubular interior for receiving the solid, a retaining device for retaining the solid in the syringe body, a needle with a second tubular interior, said needle being connected to the syringe body, and the second tubular interior of the needle communicating with the first tubular interior of the syringe body, and a ram which can be driven through the first interior into the second interior. The invention further relates to a method for injecting a solid.

### PRIOR ART

The injection of solids is a standard procedure in human and veterinary medicine and represents an alternative to the other methods of administration (e.g. oral, intravenous, etc.). A solid is injected by means of an injection device into the human or animal body. Typically, the solids are either a marker (for example for identification and/or monitoring of animals) or a solid medicament. The use of solid medicaments is routine, both in animals and humans, and has the advantage that, because of the slow absorption of the active substance, the medicament can be administered less frequently, which means that fewer consultations are needed. Applications involving the injection of solids include hormone replacement therapy and the treatment of pain.

The solid is typically injected by means of a syringe which is designed for solids and which mainly comprises a syringe body, with a needle, and a ram. After the needle has been inserted into the body, the solid is guided by means of the ram into the needle. Then, with the ram kept stationary, the needle is withdrawn from the body. The solid is retained in the body by the ram. Finally, the ram is withdrawn from the body.

DE 37 45 071 C2 (Bio Medic Data Systems) discloses a needle arrangement for marking animals. The device comprises a hollow tube with an exit opening and an entrance opening, and with a drive pin. The end of the hollow tube with the entrance opening is formed integrally in a plug that extends along part of the length of the tube. A pin with a sealing disk is used to arrange the marker near the exit opening in the hollow tube. A projection formed in one piece with the sleeve extends through the opening. This projection frictionally engages the marker until a force exerted on the plunger, and in turn on the marker, is sufficient to press the latter through the tube. A coating on the outer surface of the marker prevents the marker from falling out of the hollow tube before the marker is implanted.

U.S. Pat. No. 5,484,403 (Avid Marketing) discloses a syringe for implanting solid objects under the skin. The syringe consists of a barrel, a needle connected to the barrel, and a ram. The needle has holding means by which the object to be implanted is held until implantation, namely crimped areas on the needle wall. Alternatively, an incision can also be provided in the needle, the diameter of the needle being smaller than that of the object.

EP 0 639 387 A (Texas Instruments Inc.) discloses an injector for introducing solid objects, such as transponders, into a living being, said injector having a barrel for receiving an object, and a ram that is movable in the barrel for the purpose of ejecting the object. Clamping means for mechanically holding the object are provided in the barrel. The clamping means are designed as resilient elements. The resilient element consists of a lip made from resilient material and is arranged parallel to the longitudinal axis of the barrel. The object is ejected by means of the ram, the latter having a smaller diameter than the object, such that the axial movement thereof is not influenced by the lip.

The prior art devices for injecting solids have the disadvantage that fragile solids in particular can be damaged during the injection.

### DISCLOSURE OF THE INVENTION

The object of the invention is to make available a device that pertains to the technical field mentioned at the outset and that permits injection of a solid in a way that protects the solid.

The object is achieved by the features defined in claim 1. According to the invention, the ram is designed to cooperate directly with the retaining device, such that the retaining device can be deactivated by means of the ram.

This affords the advantage that the retaining device can be deactivated before the injection of the solid, in particular before a movement of the solid in the syringe body, with the result that, during the movement of the solid, the holding device does not subject the solid to any radial force component directed at right angles to the direction of delivery of the solid, except for the customary normal forces. During the use of the device, the solid, during its travel through the syringe body and the needle, is also not subjected to any radial force component of a retaining device, as a result of which the solid can be injected gently into a human body, for example. In this way, solids can also be injected that are brittle or deformable to some extent. Capsules, which contain liquids for example, can also be injected in this way, without any danger of the capsule being broken open by being squeezed by the retaining device. This also has the effect that the syringe is used in a way that is particularly ergonomic and not susceptible to error, since the deactivation of the retaining device takes place with the same movement as the injection of the solid.

The syringe body is preferably designed substantially as an elongate hollow cylinder. The hollow cylinder typically has a circular cylindrical interior. The needle can be made from metal, in particular steel, although plastics can also be used. The needle is preferably beveled at the distal end and is designed as a tip at the very front end, and the edges adjoining the tip can also be sharp, such that penetration into a human or animal body is made easier. The proximal end of the needle is connected to the syringe body such that the first interior of the syringe body communicates with the interior of the needle. The connection between syringe body and needle is preferably obtained by a press fit or by adhesive bonding. To protect the needle and the user, a protective cap can be provided, which can be guided over the needle prior to use and is held with a form fit and/or force fit. A safety element connected with a form fit and/or force fit to the syringe can also be provided which, in the fitted state, ensures that the ram is not accidentally pushed into the syringe body. This safety element can be designed as an elongate element which, underneath the ram head, is held thereon by means of a clip connection and thus ensures the necessary distance between the syringe body and the ram head.

The ram comprises a plunger, a ram head which lies opposite the plunger and by which the ram is handled, and a connection between the plunger and the ram head, which connection is typically designed as an elongate rod with a smaller diameter than the plunger. If appropriate, the connection can also be omitted, in which case the ram only comprises a plunger and a ram head. This is advantageous if the diameter of the plunger is very small, particularly in the millimeter range or below. The stability of the ram is thereby increased.

The injection of a solid using the syringe according to the invention preferably takes place in the following steps:
a) The needle is inserted into the human or animal body.
b) The retaining device is deactivated by means of the ram, and the solid is transferred into the needle.
c) The syringe body is drawn back, with the ram stationary in relation to the body, such that the solid outside the needle remains in the body.

The retaining device preferably comprises a retaining element, the ram being at a distance from the retaining element when the retaining device is deactivated. Before the device is used, the solid that is to be injected is positioned distally in front of the ram. The retaining element is positioned such that it can cooperate with the solid. The retaining element can now be deactivated by the ram by means of remote effect. In this way, the retaining device can be deactivated before the injection, as a result of which the solid does not have to be moved counter to a frictional force that could damage the solid. This has the advantage that the user does not have to think about the deactivation of the retaining device and can instead simply guide the ram through the syringe body. A wide variety of means can be provided for this purpose.

The ram can be connected firmly and resiliently to an elongate portion that protrudes beyond the ram in the distal direction. At the distal end of the elongate portion, a lug can be provided which, in a first state, extends through an opening into the interior of the syringe body and thus retains the solid or clamps the latter securely by friction. In this variant, the syringe body also has a ramp directly behind the lug (in the distal direction) at the edge of the opening. When the ram is driven in the distal direction, the elongate portion is lifted via the ramp out of the interior of the syringe body and thus frees the way for the solid. Of course, the elongate portion does not necessarily have to be connected securely to the ram. The elongate portion can also be guided by force, in which case the ram, for example, has a lug which can cooperate with the elongate portion. The deactivation and the injection thus take place with the same movement.

Similarly to the variant described above, the lug can be driven out of the interior of the syringe body via a ramp extending tangentially with respect to the interior of the syringe body, by means of a rotation of the ram about the axis thereof. Before the injection, the retaining device would therefore have to be deactivated by means of a rotation of the ram.

The retaining element can also be connected to the ram via pulling means, such that the ram is driven in the proximal direction before the injection, as a result of which the retaining element is pulled out of the syringe body and, during the movement of the ram in the distal direction upon injection of the solid, the retaining element no longer engages in the syringe body. This means, however, that two different movements have to be performed for the injection.

Before the deactivation of the retaining device, the solid is preferably held between the ram and the retaining element. This has the effect that, during the storage of the device, the solid is not subjected to a force caused by the retaining device and is therefore stored in a protected manner. For this purpose, in the activated state, the retaining element at least partially closes the cross section of the interior of the syringe body distally in front of the solid. When the retaining device is deactivated, the retaining element does not close the cross section, or closes it only to an extent that allows the solid to be pushed past the retaining element.

Alternatively, the retaining element can engage the solid at the side by means of frictional engagement and prevent it from accidentally slipping out of the syringe body or out of the needle.

The retaining device is preferably formed in one piece with the syringe body. This results in a syringe body, with retaining device, that is of simple construction and that can be produced inexpensively. The syringe body can also comprise several parts, but the syringe body and the retaining device are then preferably designed as one component.

Alternatively, the retaining device can be constructed as an independent component or even in several parts. This has the advantage, for example, that injection molds for producing the retaining device and the syringe body can be of a simpler design. However, more method steps have to be carried out when assembling the syringe.

The retaining element is preferably designed as at least a first lug extending into the first interior of the syringe body. With the first lug, the solid can be retained in the syringe body prior to injection. The retention can, for example, be such that the solid is arranged proximally behind the lug and thus prevented from slipping out. Moreover, the lug can also act laterally on the solid by means of frictional engagement and thus prevent the solid from slipping out. Finally, the solid could also have a depression into which the lug can extend and thereby prevent slipping of the solid in both axial directions. In this way, a retaining device could also be created in which no radial force components act on the solid. However, the solid would have to be suitably shaped for this purpose.

Instead of a lug, it is also possible to provide a retaining element that corresponds to the cross section of the first interior and that completely closes the cross section. This can be advantageous if the solid is particularly unstable. In this case, the space in which the solid is arranged can also be sealed off in a substantially airtight manner by the retaining element. However, the retaining element then has to travel a greater distance in order to free the first interior for the solid.

The first lug is preferably connected resiliently to the syringe body. This results in a particularly simple design of the retaining device. In a first state, in which the resiliency is relaxed, the first lug extends into the first interior of the syringe body. In a second state, in which a force acts on the lug in the radial direction, the first lug extends partially, or does not extend, into the first interior of the syringe body.

The resilient connection can be obtained in a variety of ways:
1. The lug can be arranged by means of a helical or conical spring, which is oriented radially with respect to the longitudinal direction of the syringe body, thereby pressing the lug into the first interior.
2. The lug can be subjected to a force by means of a flat spring, which is arranged axially or is arranged around part of the circumference of the syringe body (part-circle helical spring).
3. The lug can be subjected to a force by means of an elastic ring around the syringe body, the elastic ring being designed, for example, as a rubber band or the like.
4. Finally, the lug itself can be designed as an elastic element.

For deactivation of the retaining device, the lug can be guided out of the first interior via the resilient element and also directly by the lug itself.

Alternatively or in addition, a locking of the lug or a frictional engagement of the lug can be provided, which can be deactivated by means of the ram. A perforation in the area of the lug, which perforation is broken by a radially directed force, can also be provided as an alternative to the resilient retaining device, at any rate if the device is intended to be disposed of after one use.

The retaining device preferably comprises an elongate portion which, at a distal end, is connected to the first lug and, at a proximal end, is connected to the syringe body, in particular resiliently and/or pivotably, and the lug, in a first state, extends into the first interior of the syringe body and, in a second state, extends at most partially into the first interior of the syringe body or, in particular, does not extend into the first interior of the syringe body. This results in a simple and inexpensive design of the retaining device. For this purpose, the elongate portion itself can be made from a resilient material, or it can be designed to be pivotable at the proximal end and acted on by means of a spring. In a first state, the solid can be arranged behind the lug. The solid can also be arranged in the area of the lug and, for example, can be held by frictional engagement by the lug.

Alternatively, instead of the elongate portion, a tube can also be provided which consists, for example, of a flexible material, in particular an elastic material.

The retaining device preferably comprises two elongate portions, which lie opposite each other in relation to the first interior and which are connected at the distal end to lugs, and the elongate portions are connected at the proximal end to the syringe body, in particular resiliently and/or pivotably. The risk of the solid becoming jammed is reduced in this way.

Alternatively, it is also possible to provide only one elongate portion, in which case, however, the risk of jamming is increased. Moreover, it is also possible to provide more than two elongate portions, in which case a symmetrical arrangement around the first interior is preferably chosen. However, this means increased outlay in terms of construction and, consequently, higher production costs.

The elongate portion is preferably formed between a longitudinally oriented U-shaped recess in the syringe body. The syringe body can thus easily be formed in one piece with the retaining device, as a result of which a particularly efficient and inexpensive method of production can be achieved. Moreover, a particularly compact syringe body with a holding device can be formed in this way. For example, the syringe body can be produced, by means of a suitable mold, such that the U-shaped recess is already formed. On the other hand, the U-shaped recess can be produced by a punching operation or a milling operation after the production of the syringe body.

Alternatively, the elongate portion can also be produced separately and can be connected to the syringe body. However, this is associated with greater outlay in terms of construction and, consequently, with added costs.

The elongate portion, in the first state, is preferably oriented substantially parallel to a longitudinal direction of the first interior of the syringe body and, in the second state, is oriented in a pivoted position relative to the longitudinal direction of the first interior. If the elongate portion is designed by means of a U-shaped recess in the syringe body, this leads in a particularly simple manner to an elongate portion that is oriented parallel to the longitudinal direction of the first interior and that can be pivoted when acted on by a force. The pivoting preferably takes place radially outward in relation to the first interior. The elongate portion is in this case preferably designed to be elastic or resilient in relation to the syringe body, which can be achieved in particular by a suitable choice of material. Typically, the holding device is active in the first state and inactive in the second state. If appropriate, however, the holding device can also be active in the second state and inactive in the first state, particularly if a pivot axis is spaced radially outward from the first interior.

Alternatively, the elongate portion can also be arranged radially around at least part of the circumference of the first interior. The stability of the syringe body may be adversely affected by this.

The elongate portion preferably has, in the area of the proximal end, a second lug which, in the first state, extends into the interior of the syringe body and, in the second state, extends at most partially into the first interior of the syringe body or, in particular, does not extend into the first interior of the syringe body, and the solid, in the first state, is retained between the first lug and the second lug and, in the second state, is not retained. In this way, a retaining device is created which prevents the solid from moving in both axial directions and can fix the solid in position. Typically, the distance between the lugs is such that the solid is held between them. For this purpose, the distance has a length that exceeds the length of the solid slightly, in particular by less than 10%, preferably by less than 5%. This ensures that the solid cannot be pinched between the lugs or be damaged by the lugs. At the same time, however, a reciprocating axial movement of the solid is avoided, which movement could likewise result in damage when the solid strikes the lugs. The distance between the lugs can also be such that, with a given length tolerance of the solid upon production of the device, for example 99% or 99.9% of the solids can be held between the lugs, i.e. 99% or 99.9% of the solids have a length smaller than the distance between the lugs. However, the lugs can also be at a greater distance, such that production-related complications when introducing the solid between the lugs can be largely eliminated. However, the lugs can also be at a distance smaller than the length of the solid, in which case, for example, the solid is held laterally by means of frictional engagement.

Alternatively, it is also possible to do without the second lug, particularly if, for example, the elongate portion is connected to the ram, in which case, as a result of the movement of the ram in the distal direction, the first lug is driven out of the first interior of the syringe body via a ramp. In this case, the ram prevents the solid from moving in the proximal direction.

The ram and/or the second lug preferably in the proximal direction have a bevel such that, when the ram engages with the second lug, at least one force component can be applied radially to the second lug, and the elongate portion can be converted to the second state. The second lug designed in this way can perform two functions, namely retaining the solid in respect of the proximal direction, and the function of a lever arm, in order to pivot the elongate portion. After the elongate portion has been pivoted, the ram moves onward in the distal direction in order to convey the solid through the needle. To permit this, the second lug has a bevel. The bevel can have a constant gradient. When the ram makes contact with the bevel, the front face of the ram initially encloses an angle that increases as the ram continues to move in the distal direction. During the movement of the ram, the force that has to be applied decreases, since the angle between the front face of the ram and the bevel is increased, as also is the lever which lies between the contact area of the ram on the bevel and the pivot point. To achieve a uniform application of force, the bevel can also have a concave shape, as a result of which the increasing lever can be substantially compensated by the increasing gradient of the bevel. This means that an abrupt pushing through of the ram is avoided, and the device is thus safer to use.

Alternatively, it is also possible to do without the bevel, particularly if the second lug has a small radially measured height.

A radially measured height of the first lug is preferably greater than a radially measured height of the second lug. This can optimize the pivoting of the elongate portion. The second lug is pressed by the ram at most into the edge area of the first interior. The first lug at the same time moves out of the first interior via the elongate portion. If the second lug has the same height as the first lug, the first lug is guided radially outward further than is necessary. In this way, more force is applied than is needed for the deactivation of the retaining device. Moreover, the device may therefore be less compact during use. In order to optimize these movements, the lugs can be dimensioned such that the second lug, in the deactivated state, hardly extends into the first interior of the syringe body at all. The distance between the first lug and the first interior is preferably minimal when the retaining device is deactivated.

A ratio of the radially measured height of the first lug to a distance of the first lug from the proximal end of the elongate portion preferably lies in a range of a ratio of the radially measured height of the second lug to a distance of the second lug from the proximal end of the elongate portion. This results in an optimal size distribution of the two lugs. If the second lug is pushed out of the first interior of the syringe body by the ram, the first lug is at the same time pushed out of the first interior of the syringe body. However, in order to avoid any inaccuracies in the construction of the syringe body and of the retaining device, the first lug, in respect of the above-described ratio, is preferably made slightly smaller, in particular about 5-10% smaller, than the theoretically optimal height.

Alternatively, the first lug and the second lug can also have the same height, particularly if the lugs have a very small height or the elongate portion is relatively short.

The dimensioning of the lugs is preferably in relation to the diameter of the syringe body. The first lug and the second lug preferably have a radially measured height that is less than 50%, preferably less than 35%, particularly between 5% and 25%, of a diameter of the first interior of the syringe body. The radially measured height of the lug is particularly preferably about 20% of the diameter of the interior of the syringe body. To optimize the heights of the lugs, it must be noted that, as the height increases, the pivoting path of the elongate portions is increased and, as the height decreases, the retention of the solid may no longer be able to be guaranteed. The above sizes have proven to be optimal. The first lug and the second lug preferably have a radially measured height of approximately 0 3 mm in the case of an internal diameter of the syringe body of approximately 1.6 mm.

Alternatively, and in particular as a function of the solid that is to be injected, the dimensioning of the lug can also be chosen differently. If only one elongate portion is used, the lugs can also have a height greater than 50% of the diameter of the interior of the syringe body. In particular, the first lug could also be designed such that it substantially closes off the first interior. Accordingly, the pivoting path of the elongate portions is increased.

The ram preferably comprises a plunger, and the plunger has a length that is greater than the distance between the first lug and the second lug. This has the effect that the solid can be held between the lugs. Thus, setting aside its own force of gravity, and without force being applied, the solid can be prevented from slipping out of the syringe body.

Alternatively, the distance can also be smaller. In this case, the solid would be held by a frictional force between the lug (or the lugs) and the solid. With a suitable surface of the lug and/or of the solid, the force applied could be relatively small, such that the advantages of the gentle handling of the solid would nevertheless be achieved, particularly since the solid would be moved by the ram only when the holding device is deactivated. For this purpose, the lug could have a surface structure that provides a particularly high coefficient of friction between the solid and the lug. Moreover, the lug could also have a suitable coating (e.g. rubber) or be made from a suitable material.

The ram preferably has recesses which cooperate with the first lug and/or the second lug and prevent the ram from being pulled back in the proximal direction. This can be achieved, for example, if the ram has grooves oriented transversely with respect to the longitudinal direction and if the second lug is wedge-shaped, such that the second lug can come into engagement with the grooves. Since the bevel of the second lug is oriented in the proximal direction and the distal side of the wedge-shaped lug is oriented radially, that is to say at right angles to the longitudinal direction, the ram can be moved only in the distal direction. In order to optimize this effect, the grooves can be formed as wedge-shaped recesses, in which case a side oriented at right angles to the longitudinal direction is arranged distally.

Alternatively, it is also possible to do without a design of the ram as described above, particularly if the syringe is intended to be used more than once.

The syringe body is preferably made from a resilient material, in particular a metal, a metal alloy or a plastic. This permits a one-piece design of the syringe body with the retaining device. The demands in terms of the elastic nature of the material are not especially high. The pivoting of the resilient element is typically quite small. The pivoting angle of the elongate portion is preferably less than 10°, preferably less than 5°, in particular less than 2°.

Alternatively, the syringe body can also be made from a non-resilient material, particularly if the retaining device is not formed in one piece with the syringe body.

The syringe body is preferably made from a transparent plastic. In this way, the user is able to visually monitor the administration status of the solid. The plastic used can be, for example, polymethyl methacrylate (PMMA), polycarboate, or other plastics known to a person skilled in the art. Although PMMA is relatively brittle and only slightly elastic, this plastic can be used for the production of the syringe body, particularly since there are no strict demands on the elastic property.

Alternatively, it is also possible to use non-transparent materials. It is also conceivable to provide a viewing window in the syringe body, which window is made from a material different than that of the syringe body.

However, a large number of suitable plastics are known to a person skilled in the art. Thus, other suitable plastics could also alternatively be used that have a certain degree of elasticity and a sufficiently high strength.

Further advantageous embodiments and combinations of features of the invention will become clear from the following detailed description and from all of the patent claims.

### BRIEF DESCRIPTION OF THE DRAWINGS

In the drawings used to explain the illustrative embodiment:

FIG. 1 shows a schematic view of a device according to the invention for injecting a solid;

FIG. 2 shows a schematic view of a cross section along a longitudinal axis of the device for injecting a solid, prior to use;

FIG. 3 shows a schematic view of a cross section along a longitudinal axis of the device for injecting a solid, with the retaining device deactivated;

FIG. 4 shows a schematic view of a cross section along a longitudinal axis of the device for injecting a solid, with the retaining device deactivated and with the solid positioned in the needle;

FIG. 5 shows a schematic view of a cross section along a longitudinal axis of another possible embodiment of a device according to the invention for injecting a solid, with the device inserted into a body and prior to the actuation of the ram;

FIG. 6 shows a schematic view of a cross section along a longitudinal axis of the device for injecting a solid, with the retaining device deactivated and with the solid positioned in the needle, which needle is inserted into a body; and

FIG. 7 shows a schematic view of a cross section along a longitudinal axis of the device for injecting a solid, after the solid has been positioned in the body.

In principle, the same parts in the figures are provided with the same reference signs.

### WAYS OF IMPLEMENTING THE INVENTION

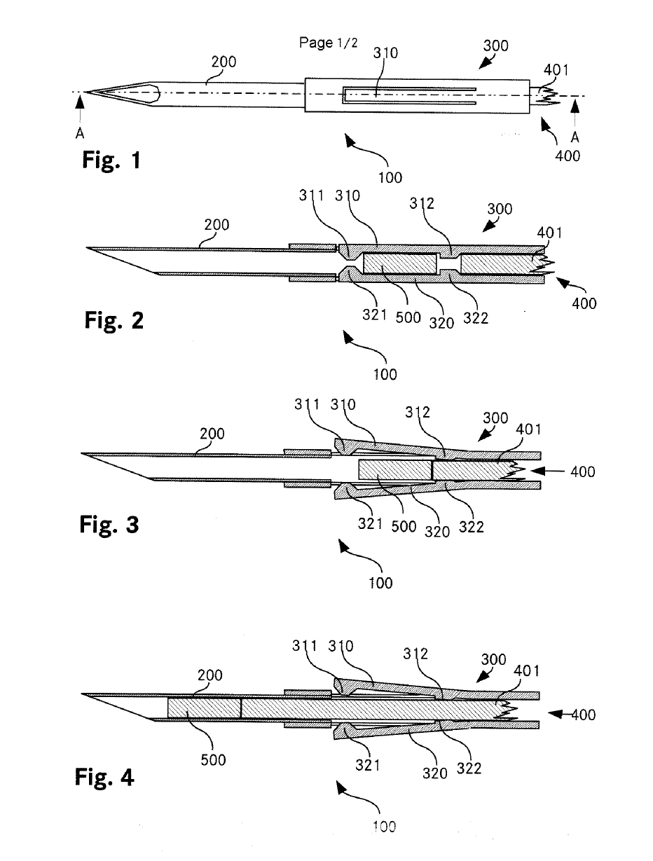

FIG. 1 shows a schematic view of a device 100 for injecting a solid 500, which device 100 comprises a needle 200, a syringe body 300 and a ram 400. The needle 200 has a bevel at a distal end, so as to facilitate insertion into a body. The syringe body 300 is designed substantially as a circular cylinder. Along a longitudinal axis, the syringe body has two mutually opposite U-shaped recesses, which enclose an elongate, resilient portion 310, with FIG. 1 showing only one U-shaped recess. The needle 200 is connected by a proximal end to the syringe body 300. The ram 400 comprises a plunger 401 and a ram head (not shown). The plunger 401 has substantially the shape of a circular cylinder. The interiors of the syringe body 300 and of the needle 200 have a corresponding shape, such that the ram 400 can be driven through the syringe body 300 and the needle 200. The syringe body 300 can comprise a protective sheath (not shown), which can avoid entry of contaminants at the U-shaped recess. The protective sheath is either flexible or has recesses in the area of the elongate portions 310, 320, such that the pivoting of the elongate portions 310, 320 is not impeded.

The syringe body 300 and the ram 400 are made from a plastic, in particular from a transparent plastic, for example from polymethyl methacrylate (PMMA) or polycarbonate. The needle 200 is made from metal, in particular from steel.

FIG. 2 shows a schematic view of a cross section, along the axis A-A in FIG. 1, of a device 100 for injecting a solid 500, prior to use. The needle 200 can be seen, which is connected to the syringe body 300. In the present embodiment, the syringe body 300 comprises two mutually opposite elongate portions 310; 320, which each comprise a first lug 311; 321 and a second lug 312; 322, which extend into the interior of the syringe body 300. The elongate portions 310; 320 are arranged parallel to the longitudinal axis of the syringe body 300 and, at the proximal end, are connected resiliently and pivotably in the syringe body 300. In a state with no force applied, the elongate portions 310; 320 are oriented parallel to the longitudinal axis. The first lugs 311; 321 are arranged at a distal end of the corresponding elongate portion 310; 320 and connected thereto. The second lugs 312; 322 are arranged in the area of the proximal end of the corresponding elongate portion 310; 320 and connected thereto. The solid 500 has the shape of a circular cylinder, which has a diameter in the range of the diameter of the plunger 401. In the present embodiment, the distance between the first lugs 311; 321 and the second lugs 312; 322 is slightly greater than the length of the solid 500, such that the solid is held with a form fit between the first lugs 311; 321 and the second lugs 312; 322 and is thereby prevented from moving in a longitudinal direction. The second lugs 312; 322 are arranged at a distance distally from the connection in the syringe body 300, such that a lever action can be achieved when the ram 400 makes contact with the second lug 312; 322. The second lugs 312; 322 for this purpose have a bevel in the proximal direction. When the ram 400 is moved in the distal direction, the ram 400 makes contact with the bevel of the second lugs 312; 322, in order to drive them radially outward.

FIG. 3 shows a schematic view of a cross section along a longitudinal axis of a device 100 for injecting a solid 500, with the retaining device deactivated, the same elements being shown as in FIG. 2. The ram 400 has been advanced in the distal direction. The jacket surface of the ram 400 makes contact with the second lugs 312; 322, such that these lugs are pivoted radially outward in relation to FIG. 2. The first lugs 311; 321 connected to the second lugs 312; 322 via the elongate portion 310; 320 are, at the same time and by remote effect, guided radially outward by means of the interaction of the second lugs 312; 322 and of the plunger 401, as a result of which the first lugs 311; 321 no longer extend (or extend at most partially) into the interior of the syringe body 300 and thus free the way for the solid 500 through the interior of the syringe body 300 and into the needle 200.

FIG. 4 shows a schematic view of a cross section along a longitudinal axis of a device 100 for injecting a solid 500, with the retaining device deactivated and with the solid positioned in the needle. The elements shown correspond to those in FIGS. 2 and 3, the plunger 401 of the ram 400 having been advanced into the needle 200, as a result of which the solid 500 comes to lie in the needle 200.

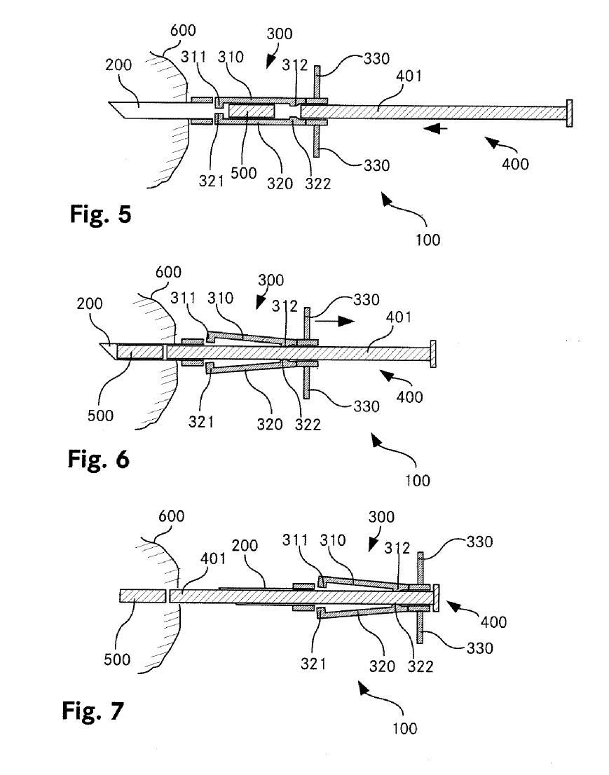

FIG. 5 shows a schematic view of a cross section along a longitudinal axis of a device 100 for injecting a solid 500, with the device inserted into a body 600 in the state prior to the actuation of the ram 400. The elements shown here correspond largely to those in FIGS. 2-4. In contrast to the preceding figures, FIG. 5 also shows a holding element 330, which is arranged in the proximal area of the syringe body 300 and connected thereto. In the present embodiment, the holding element 330 is designed circumferentially and can be oval or kidney-shaped, for example.

The needle 200 is inserted up to the distal end of the syringe body 300 into a body 600 (of an animal or a human). The device 100 is thus ready for the injection. In a subsequent step, the ram 400 is driven into the syringe body, whereupon the ram 400, at the second lugs 312, 322, drives the elongate portions 310, 320 radially outward, as a result of which the first lugs 311, 321 are also driven radially outward in proportion to the radial movement of the second lugs 312, 322. In this way, the path of the solid 500 through the syringe body 300 and into the needle 200 is freed. It should be noted here that the plunger 401 does not convey the solid 500 into the body, but only into a distal area of the needle 200 located in the body. This avoids damage to the body tissue by the mostly blunt solid 500 and causes the least possible pain.

FIG. 6 shows a schematic view of a cross section along a longitudinal axis of a device 100 for injecting a solid 500 according to FIG. 5. The retaining device is deactivated, and the solid 500 is already positioned by the plunger 401 in the needle, which is located within the body 600. In particular, the solid 500 does not protrude from the needle 200, such that the tissue of the body 600 is not damaged by the mostly blunt solid. The elongate portions 310, 320 are pivoted radially outward. In this state, the plunger 401 is not driven completely into the syringe body 300 but instead protrudes from the proximal end of the syringe body 300 by at least a length that corresponds to the depth of insertion of the needle 200 in the body. The exact length by which the ram 400 is to be driven into the syringe body 300 can, for example, be simply marked on the plunger 401 or can be formed as a notch in the plunger 401, such that one or both of the second lugs 312, 322 catches reversibly as soon as the ram 400 is driven sufficiently far into the needle 200 or through the syringe body 300.

In a subsequent step, the syringe body is gripped by the holding element 330 and drawn back in the proximal direction over the ram 400, which is stationary relative to the body 600, until the needle 200 moves over the solid 500 and thus leaves the solid 500 behind in the body 600. By virtue of the fact that the ram 400 remains stationary during the withdrawal of the syringe body 300, the solid 500 is not pulled back out of the body by frictional forces created by the inner wall of the needle 200 during the withdrawal of the needle 200.

FIG. 7 shows a schematic view of a cross section along a longitudinal axis of a device 100 for injecting a solid 500, after the solid 500 has been positioned in the body 600. The syringe body 300 has been drawn back completely in relation to the ram 400, such that the needle 200 no longer extends into the body 600. In this state, the plunger 401 is still located in the body 600. In a final step, the device 100 is drawn back from the body 600, such that the distal end of the plunger 401 is removed from the body 600.

In principle, the device 100 can given any desired dimensions and, in particular, these dimensions can be adapted to the solid 500 that is to be injected.

The solid 500 can also have another shape, without this adversely affecting the function of the device. For example, the solid 500 can have the shape of a pellet or a ball. Other shapes are also conceivable. However, the smallest geometric projection of the solid can preferably be inscribed in the cross-sectional surface area of the interior of the syringe body.

The distance between the first lugs 311; 321 and the second lugs 312; 322 can also be smaller than a length of the solid, as a result of which the solid is held by frictional engagement between the first lugs 311, 321.

The bevel of the second lugs 312; 322 can also be omitted, particularly if the plunger 401 is rounded at the distal end or, correspondingly, has a cross section decreasing in the distal direction, i.e. is beveled.

The holding element 330 can also be designed in two or more parts. For example, two mutually opposite elements could be provided, such that the syringe body 300 can be held between index finger and middle finger, and the ram 400 can be actuated using the thumb.

In conclusion, the invention makes available a device for injecting a solid, said device permitting injection of a solid in a way that protects the solid.

1. Device (100) for injecting a solid (500) into a human or animal body, in particular a syringe for injecting a solid medicament, said device (100) comprising:
a) a syringe body (300) with a first tubular interior for receiving the solid (500);
b) a retaining device for retaining the solid (500) in the syringe body (300);
c) a needle (200) with a second tubular interior, said needle (200) being connected to the syringe body (300), and the second tubular interior of the needle (200) communicating with the first tubular interior of the syringe body (300);
d) a ram (400), which can be driven through the first interior into the second interior;
characterized in that
e) the ram (400) is designed to cooperate directly with the retaining device, such that the retaining device can be deactivated by means of the ram (400).

2. Device (100) according to claim 1, characterized in that the retaining device comprises a retaining element (311; 321), the ram (400) being at a distance from the retaining element (311; 321) upon deactivation of the retaining device.

3. Device (100) according to claim 2, characterized in that the solid (500) is held between the ram (400) and the retaining element (311; 321) prior to the deactivation of the retaining device.

4. Device (100) according to claim 1, characterized in that the retaining device is formed in one piece with the syringe body (300).

5. Device (100) according to claim 1, characterized in that the retaining element (311; 321) is designed as at least a first lug (311; 321) extending into the first interior of the syringe body (300).

6. Device (100) according to claim 5, characterized in that the first lug (311; 321) is connected resiliently to the syringe body (300).

7. Device (100) according to claim 5, characterized in that the retaining device comprises an elongate portion (310; 320) which, at a distal end, is connected to the first lug (311; 321) and, at a proximal end, is connected to the syringe body (300), in particular resiliently and/or pivotably, and the lug (311; 321), in a first state, extends into the first interior of the syringe body (300) and, in a second state, extends at most partially into the first interior of the syringe body (300) or, in particular, does not extend into the first interior of the syringe body (300).

8. Device (100) according to claim 7, characterized in that the retaining device comprises two elongate portions (310; 320), which lie opposite each other in relation to the first interior and which are connected at the distal end to lugs (311; 321), and the elongate portions (310; 320) are connected at the proximal end to the syringe body (300), in particular resiliently and/or pivotably.

9. Device (100) according to claim 7, characterized in that the elongate portion (310; 320) is formed between a longitudinally oriented U-shaped recess in the syringe body (300).

10. Device (100) according to claim 7, characterized in that the elongate portion (310; 320), in the first state, is oriented substantially parallel to a longitudinal direction of the first interior of the syringe body (300) and, in the second state, is oriented in a pivoted position relative to the longitudinal direction of the first interior.

11. Device (100) according to claim 7, characterized in that the elongate portion (310; 320) has, in the area of the proximal end, a second lug (312; 322) which, in the first state, extends into the interior of the syringe body (300) and, in the second state, extends at most partially into the first interior of the syringe body (300) or, in particular, does not extend into the first interior of the syringe body (300), and the solid (500), in the first state, is retained between the first lug (311; 321) and the second lug (312; 322) and, in the second state, is not retained.

12. Device (100) according to claim 11, characterized in that the ram (400) and/or the second lug (312; 322) in the proximal direction have a bevel such that, when the ram (400) engages with the second lug (312; 322), at least one force component can be applied radially to the second lug, and the elongate portion (310; 320) can be converted to the second state.

13. Device (100) according to claim 11, characterized in that a radially measured height of the first lug (311; 321) is greater than a radially measured height of the second lug (312; 322).

14. Device (100) according to claim 11, characterized in that a ratio of the radially measured height of the first lug (311; 321) to a distance of the first lug from the proximal end of the elongate portion (310; 320) lies in a range of a ratio of the radially measured height of the second lug (312; 322) to a distance of the second lug (312; 322) from the proximal end of the elongate portion (310; 320).

15. Device (100) according to claim 11, characterized in that the first lug (311; 321) and the second lug (312; 322) have a radially measured height that is less than 50%, preferably less than 35%, particularly between 5% and 25%, of a diameter of the first interior of the syringe body (300).

16. Device (100) according to claim 11, characterized in that the ram (400) comprises a plunger (401), and the plunger (401) has a length that is greater than the distance between the first lug (311; 321) and the second lug (312; 322).

17. Device (100) according to claim 11, characterized in that the ram (400) has recesses which cooperate with the first lug (311; 321) and/or the second lug (312; 322) and prevent the ram (400) from being pulled back in the proximal direction.

18. Device (100) according to claim 1, characterized in that the syringe body (300) is made from an at least slightly resilient material, in particular a metal, a metal alloy or a plastic.

19. Device (100) according to claim 1, characterized in that the syringe body (300) is made from a transparent plastic.

20. Method for injecting a solid (500) into a human or animal body using a device (100) that comprises a syringe body (300) for receiving the solid (500), a needle (200) connected to the syringe body (300), a retaining device, and a ram (400) mounted movably in the syringe body (300), in particular using a device (100) according to claim 1, said method being characterized by the following steps:
a) The needle (200) is inserted into the human or animal body (600).
b) The retaining device is deactivated by means of the ram (400), and the solid (500) is transferred into the needle (200).
c) The syringe body (300) is drawn back, with the ram (400) stationary in relation to the body (600), such that the solid (500) outside the needle (200) remains in the body.
